(19) United States
(12) Patent Application Publication
(10) Pub. No.: US 2012/0197060 A1
(43) Pub. Date: Aug. 2, 2012

(54) RADIATION ENHANCED MACROMOLECULAR DELIVERY OF THERAPEUTIC AGENTS FOR CHEMOTHERAPY TECHNOLOGY

(75) Inventors: Abhijit Ray, Bhubaneswar (IN); Hamidreza Ghandehari, Salt Lake City, UT (US)

(73) Assignee: UNIVERSITY OF UTAH RESEARCH FOUNDATION, Salt Lake City, UT (US)

(21) Appl. No.: 13/378,731

(22) PCT Filed: Jun. 17, 2010
(86) PCT No.: PCT/US10/38932

§ 371 Date: Apr. 20, 2012

Related U.S. Application Data
(60) Provisional application No. 61/218,256, filed on Jun. 18, 2009.

(51) Int. Cl. A61M 37/00; A61P 35/00; C07F 15/00; C08F 222/38; C07H 23/00

(52) U.S. Cl. 600/1; 525/328.4; 536/6.4; 546/4

(57) ABSTRACT

Described herein are anti-cancer compounds composed of a macromolecule comprising (1) at least one anti-cancer agent directly or indirectly bonded to the macromolecule and (2) at least one high Z element directly or indirectly bonded to the macromolecule that is capable of producing Auger electrons upon exposure to X-ray energy. When the compounds are exposed to low energy X-ray (e.g., kilo electron volts KeV) Auger electrons are produced by the high Z elements present in the compound. Because lower energy is required when compared to typical radiotherapy, which uses therapeutic X-ray energy in the million electron volt range (MeV), the subject experiences lower collateral damage when compared to radiation therapy. Additionally, the presence of the anti-cancer agent provides a second mechanism for killing cancer cells. Methods for making and using the anti [symbol] cancer compounds are also described herein.

### CROSS REFERENCE TO RELATED APPLICATIONS

This application claims priority upon U.S. provisional application Ser. No. 61/218,256, filed Jun. 18, 2009. This application is hereby incorporated by reference in its entirety for all of its teachings.

### BACKGROUND

According to the CDC's National Center for Health Statistics, cancer is the second leading cause of death within the United States. Approximately 560,000 people die each year from this disease, and billions of dollars are spent providing healthcare and treatment to afflicted individuals.

Over the last half century, progress has been made for treating various cancers. For example, numerous drugs have been developed. A non-exhaustive list of these drugs includes cisplatin, carboplatin, docetaxel, paclitaxel, gemcitabine, and 5-fluorouracil. These drugs have various modes of action such as inducing DNA breaks, inhibiting DNA replication, and inhibiting transcription. However, dose limiting toxicity has limited the success of treatment modalities with existing chemotherapeutic agents. While these drugs slow tumor cell growth, collateral damage is high. Failure of therapy is due to the indiscriminate biodistribution of the employed agents in nontarget tissues. Many of these drugs not only act to slow tumor growth, but they also indiscriminately harm healthy rapidly dividing cells. Further poor water solubility has resulted in low accumulation of therapeutic agents inside the tumor. To counteract this problem, researchers have attempted to link drugs to a macromolecular conjugates including water soluble polymers. In theory, these macromolecular conjugates aid in targeting tumor cells by enhanced permeability and retention effect (EPR) and function to limit additional collateral cellular damage (Alexis et al Urol Oncol 26, 74-85. (2008), Langer, Nature 392, 5-10. (1998)). In addition to these treatments, many clinicians use high energy radiation either as a primary treatment or as an adjuvant to the drugs mentioned above. Radiation therapy also causes a large amount of collateral damage to healthy cells frequently limiting its usage. In spite of improvements in cancer treatment, the overall survival rate of cancer patients has not drastically improved.

### SUMMARY

Described herein are anti-cancer compounds composed of a macromolecule comprising (1) at least one anti-cancer agent directly or indirectly bonded to the macromolecule (2) at least one high Z element directly or indirectly bonded to the macromolecule that is capable of producing Auger electrons upon exposure to X-ray energy and (3) a targeting group to target the tumor or the angiogenic blood vessels directly or indirectly bonded to the macromolecule. The macromolecule-drug-high-Z-element conjugate is capable of selective accumulation in the tumor. When the compounds are exposed to low energy X-ray (e.g., at the high Z element's K or L edge ionization energy frequently in kilo electron volts (KeV) or lower) lethal Auger electrons are produced in short range (in hundreds of nanometers to a few microns) by the high Z elements present in the compound resulting in the break of double strand of DNA. The subject experiences lower energy (in the range of diagnostic X-ray) when compared to typical radiotherapy, which uses therapeutic X-ray energy in the million electron volt range (MeV), resulting in lethal damage to tumor tissue but lower collateral damage when compared to radiation therapy. Additionally, the presence of the anti-cancer agent provides a second mechanism for killing cancer cells. Methods for making and using the anti-cancer compounds are also described herein.

The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows a schematic drawing of G0, G1, G2 and higher generation dendrimers showing the tree-like branching architecture and the controlled incremental increase in size, molecular weight and number of surface groups with the increase in generation (G) number. These dendrimers are attached with a targeting moiety, a drug and a high Z element.

FIG. 2 shows the structure of a linear polymer attached with targeting moiety, an anticancer drug and a high Z element. The high Z element can be in any oxidation state. The high Z element can be either smaller or larger than the polymer.

FIG. 3 shows the polymer in the form of micelles or liposomes that can entrap drugs and high Z elements with a targeting group attached to the surface.

FIG. 4 shows the mechanism of producing an Auger electron from platinum.

FIG. 5 shows the irradiation of Pt with X-ray produces Auger electrons, which causes double strand DNA break within few millimeters from its origin.

FIG. 6 shows a prophetic synthetic scheme of Fmoc-GFLG-ONp.

FIG. 7 shows a prophetic synthetic scheme of dendrimer-GFLG-ethylenediamine-Pt-GFLG-SN38 conjugates.

FIG. 8 shows a prophetic synthetic scheme of N-(2-hydroxypropyl)methacrylamide (HPMA) copolymer attached with two drug combination by an enzymatically degradable tetrapeptide GFLG. The first drug is cisplatin and the second drug is docetaxel. The polymer is attached to a targeting moiety by the enzymatically non-degradable dipeptide GG.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a cell cycle specific compound” includes mixtures of two or more such compounds, and the like.

“Optional” or “optionally” means that the subsequently described event or circumstance can or cannot occur, and that the description includes instances where the event or circumstance occurs and instances where it does not. For example, the phrase “optionally a cell cycle specific compound” means that the compound can or can not be included.

The term “bonded” refers to either chemical bonding (e.g., covalent or non-covalent bonding such as hydrogen bonding, dipole-dipole interactions, electrostatic, etc.) or the process of encapsulation or entrapment

Described herein are compounds composed of a macromolecule including (1) at least one anti-cancer agent directly or indirectly bonded to the macromolecule and (2) at least one high Z elements directly or indirectly bonded to the macromolecule that is capable of producing Auger electrons upon exposure to X-ray energy. These macromolecules are capable of passively targeting tumor cells and tissues to reduce or prevent tumor cell proliferation. In certain aspects, the macromolecule optionally has a targeting moiety. The targeting moiety can actively target either the tumor or the angiogenic blood vessel. Such targeting can be specific to antigens, growth factors, tumor promoters, essential hormones, enzymes or nutrients. Cell proliferation is reduced or prevented by applying radiation-ray irradiation at the ionization energy of the K or L edge electrons of the high Z elements present to the tumor in the presence of the compounds described herein. Additionally, the presence of an anti-cancer agent on the compound also kills cancer cells and prevents their proliferation. In certain aspects, the high Z elements can also be an anticancer drug e.g. platinum in carboplatin or cisplatin. Thus, the compounds described herein provide dual mechanisms for killing cancer cells. FIG. 2 shows an exemplary structure of a linear polymer attached with targeting moiety, an anticancer drug and a high Z element. The high Z element can be in any oxidation state. The high Z element can be either smaller or larger than the polymer.

In one aspect, the compound comprises the formula I

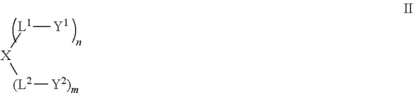

wherein
X is a macromolecule;
L¹ and L² are a linker;
Y¹ comprises a high Z elements group bonded to the linker L¹;
Y² comprises an anti-cancer agent covalently bonded to the linker L²;
m and n are, independently, an integer from 1 to 10,000; and
when present, Y³ comprises a targeting agent covalently bonded to the linker L³.

The selection of the macromolecule can vary; however, it should preferentially facilitate the uptake of the compound by cancer cells. For example, the compounds described herein can accumulate inside a tumor via the enhanced permeability and retention (EPR) effect. EPR is the passive accumulation of substances such as macromolecular conjugates inside a tumor. This property is associated with a compound's affinity for accumulating in tumor tissue much more rapidly than in normal tissues. For tumor cells to grow quickly, blood vessel production must be stimulated. Newly formed tumor blood vessels are usually abnormal in form and architecture. For example, tumor blood vessels display poorly-aligned endothelial cells with wide fenestrations, and tumor cells and tumor tissues generally lack effective drainage. Due to these defects and the presence of tumor vascular permeability factor, bradykinin, and tumor necrosis factor, tumor vasculature permits large macromolecules to enter tumor tissue more quickly than into normal tissues. In addition, poor lymphatic drainage and high hydrostatic pressure results in delayed clearance and longer retention of macromolecules within tumors.

A variety of macromolecules are suitable for use herein and generally include any macromolecule that is biocompatible, e.g., non-toxic and non-immunogenic. In certain aspects, the macromolecule is synthetic so that the molecular weight range to achieve a size appropriate for enhanced trans-endothelial permeation and retention at a tumor site and for renal filtration. In another aspect, two or more biocompatible macromolecules of molecular weight less than 45 KDa or size less than 8 nm (nanometers) can be joined by linkers. The linkers may either be biodegradable or non-bio-degradable. In certain aspects, one or more of these units can also be organic or inorganic nanoparticles. In the presence of more than one nanoparticle, the second nanoparticle may or may not be a high Z element. It may also be desirable that the macromolecules are hydrophilic in order to produce water-soluble pharmaceuticals. Finally, the macromolecule should also be stable, particularly after the high Z element group and anti-cancer agent have been bonded to the macromolecule.

The molecular weight of the macromolecule can vary. By varying the molecular weight of the macromolecule, it is possible to modify the blood circulation lifetime and body distribution of the compound, in particular its enhanced endothelial extravasation and retention at the tumor. The polydispersity of the macromolecule is also a factor in circulation lifetime and distribution. In one aspect, the macromolecule has a molecular weight of between about 1 kD to 5,000 kD, 5 kD to 500 kD, or 10 kD to 200 kD.

The size (hydrodynamic volume) of the macromolecule can vary. By varying the size (hydrodynamic volume) of the macromolecule, it is possible to modify the blood circulation time and body distribution of the compound, in particular its enhanced endothelial extravasation and retention at the tumor. The polydispersity of the macromolecule is also a factor in circulation time and distribution. In one aspect, the macromolecule has a hydrodynamic volume of between about 0.1 nm (nanometer) to 5,000 nm, 1 nm to 1000 nm, or 5 nm to 500 nm.

Macromolecules suitable for in vivo administration include, but are not limited to, dextran, dextrin, hyaluronic acid, chitosan, polylactic/glycolic acid (PLGA), poly lactic acid (PLA), polyglutamic acid (PGA), polymalic acid, polyaspertamides, polyethylene glycol (PEG), N-(2-hydroxypropyl)methacrylamide (HPMA), polyvinylpyrrolidone, polyethyleneimine, polyamidoamine (linear), and dendrimers comprising polyamidoamine, polypropyleneimine, polyether, polylysine, or any combination thereof. In another aspect, the macromolecule includes N-alkyl acrylamide macromolecules such as homopolymers and copolymers prepared from monomers of the acrylamide family including acrylamide, methacrylamide and hydroxypropylacrylamide.

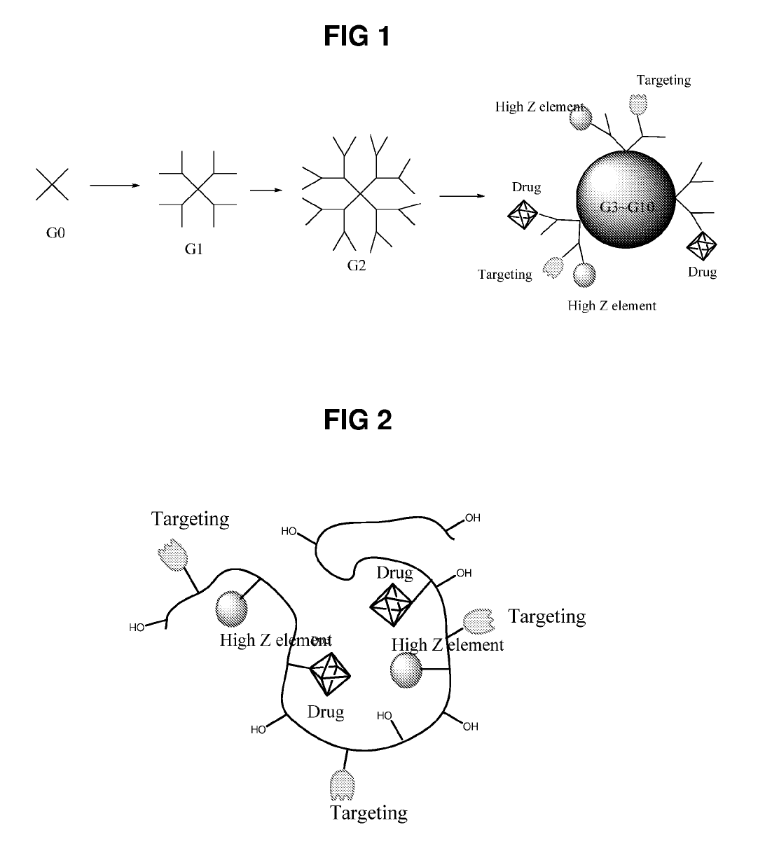

In one aspect, the macromolecule can be a dendrimer. Dendrimers are multi-functional, macromolecular, symmetric, nano-sized macromolecules useful as delivery devices. They are characterized by a unique tree-like branching architecture and a compact spherical shape in solution. FIG. 1 depicts several structures of dendrimers, where the G value is the number of generations present in the dendrimer. Their potential as drug carriers arises from the large number of arms and surface groups that can be functionalized to immobilize drugs, enzymes, targeting moieties, or other bioactive agents. The molecular weight of the dendrimer can be adjusted with appropriate linkers and drugs. The use of dendrimers herein can provide several unique features with respect to the delivery of drugs, including (ii) a dendrimer's architecture can dramatically influence pharmacokinetics; (iii) the addition of certain groups such a, for example, PEGylation, increases water solubility and dendrimer size, and can lead to improved retention and biodistribution characteristics; (iv) therapeutic agents can be internalized in the void space between the periphery and core, or covalently attached to functionalized surface groups; and (v) targeting moieties can be bound to the dendrimer's surface (discussed below). In one aspect, the dendrimer includes poly generation 1, 1.5, 2, 2.5. 3, 3.5, 4, 4.5, 5, 5.5, 6, 6.5, 7, or 7.5, 8, 8.5, 9, 9.5, 10. The dendrimer can be produced from a variety of different building blocks. In one aspect, the macromolecule is poly(amido amine) (PAMAM), diaminobutane (DAB), diaminoethane (DAE), melamine based or poly (ethylene glycol) derived.

In another aspect, the macromolecule can be a water soluble drug delivery system including an inert synthetic polymeric carrier. In this aspect, the macromolecule is 5.0 to 99.5 mol % monomeric units including, but not limited to, N-(2-methylpropyl)methacrylamide, N-(2-methylethyl)methacrylamide, N-isopropyl methacrylamide, N,N-dimethacrylamide, N-vinylpyrrolidone, vinyl acetate, 2-methacryloxyethyl glycoside, acrylic acid, methacrylic acid, vinylphosphonic acid, styrenesulfonic acid, malic acid, 2-methacryloxyethyltrimethylammonium chloride, 2-methacrylamidopropyltrimethylammonium chloride, methacryloylcholine methyl sulfate, 2-methacryloxyethyltrimethylammonium bromide, 2-vinyl-1-methylpyridinium bromide, 4-vinyl-1-methylpyridinium bromide, ethyleneimine, (N-acetyl)ethyleneimine, (N-hydroxyethyl)ethyleneimines, allylamine, or any combination thereof.

In yet another aspect, the water soluble drug delivery system based on monomers of N-(2-hydroxypropyl)methacrylamide (HPMA) or any other related monomers, may be used as universal vehicles for the specific delivery of high Z elements or anticancer agent. Without wishing to be bound by in theory, in certain aspects, any of these agents can be covalently loaded onto these delivery systems via biodegradable or non-biodegradable spacers resulting in greatly improved pharmacokinetics and better water solubility of loaded drugs.

In one aspect, two strategies can be used for introducing functional moieties into the N-(2-hydroxypropyl)methacrylamide (HPMA) copolymers: (1) copolymerization of HPMA with polymerizable functional comonomers; and (2) direct conjugation of active ester containing HPMA copolymers with functional moieties, which have a primary amine group.

In certain aspects, the macromolecule conjugate can include a recurring unit of formula (II) and a recurring unit of formula (III) as set forth herein, wherein: each A¹ and A² in formula (II) and (III) can be independently oxygen or NR³, wherein R³ can be hydrogen or CM alkyl; R¹ can be a compound that includes a drug; and R² can be a compound that can include an agent, a polydentate ligand or a polydentate ligand precursor with protected oxygen atoms, wherein the agent can be selected from high Z elements.

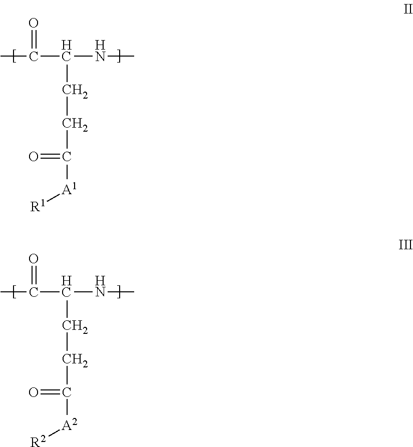

A “polydentate ligand” is a ligand that can bind itself through two or more points of attachment to a metal ion through, for example, coordinate covalent bonds. Examples of polydentate ligands include, but are not limited to, diethylenetriaminepentaacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE), 2,4-pentanedione (acac), and ethanedioate (ox).

A “polydentate ligand precursor with protected oxygen atoms” is a polydentate ligand comprising oxygen atoms, such as the single-bonded oxygen atoms of carboxyl groups that are protected with suitable protecting groups. Suitable protecting groups include, but are not limited to, lower alkyls, benzyls, and silyl groups.

The anti-cancer agent and high Z elements can be directly or indirectly bonded to the macromolecule. The term “indirectly bonded” as used herein is defined when the anti-cancer agent and/or high Z element is attached to the macromolecule via a linker Conversely, the term “directly bonded” as used herein is when the anti-cancer agent and/or high Z elements are attached to the macromolecule without a linker. In the case of the anti-cancer agent, the agent is generally covalently attached to the linker (i.e., indirect bonding) or macromolecule (i.e., direct bonding). As will be discussed below, the high Z element group can be bonded to the linker or macromolecule carrier in a number of different ways depending upon the selection of the high Z element and functional groups present on the linker or macromolecule. In general, the macromolecule has one or more functional groups that can form a covalent bond with the linker. Similarly, the linker contains functional groups that can (1) form a covalent bond with the macromolecule and (2) form a bond (covalent or non-covalent) with the high Z element and a covalent bond with the anti-cancer agent. Methods for producing the compounds described herein are provided below.

The nature and selection of the linker can vary. As discussed above, the linker can include one or more functional groups that are capable of forming covalent bonds with the macromolecule as well as form a bond with the high Z element group and anti-cancer agent. The functional groups generally contain heteroatoms such as oxygen, nitrogen, sulfur, or phosphorous. Examples of functional groups present on the linker include, but are not limited to, hydroxyl, carboxyl (acids, esters, salts, etc.), amide, amino (substituted and unsubstituted), thiol, acyl hydrazones and the like.

The selection of the linker can also vary one or more properties of the compound. For example, the linker can be a group that modifies the hydrophobic or hydrophilic properties of the compound. An example of this is polyethylene glycol (PEG). PEG is generally a hydrophilic material, and by varying the molecular weight of PEG, the hydrophilic properties of the compound can be modified. In one aspect, PEG has a molecular weight from 50 D to 200 kD, 50 D to 100 kD, 50 D to 50 kD, or 50 D to 20 kD. PEG can also be used to produce biocompatible copolymers such as, for example, (PEG-diacrylate (PEGDA), PEG-dimethacrylate (PEGDM), PEG-diacrylamide (PEGDAA), or PEG-dimethacrylamide (PEGDMA). Although PEG and related compounds are suitable as a linker herein, the linker can be other groups such as, for example, short chain (e.g., C₁-C₆) ethers, esters, amines, amides, and the like.

In other aspects, the linker can be an oligopeptide sequence, an amino acid, or amino acid sequence Amino acids can be useful as the linker when they are used to bind the high Z element to the macromolecule. For example, amino acids can contain amino, thiol, and carboxyl groups that can form non-covalent bonds with high Z elements. In this aspect, the high Z element is non-covalently bonded to the linker via coordinate covalent bonding. The functional groups present on the amino acid or oligopeptide also permit attachment of the linker to the macromolecule. In one aspect, the amino acid or oligopeptide linkers are 1 to 6 amino acids in length. In this aspect, the amino acid or oligopeptide linkers include, but are not limited, to the following sequences: Gly-Ileu-Phe, Gly-Val-Phe, Gly-Gly-Phe, Gly-Gly-Phe-Phe, Gly-Ileu-Tyr, Phe, Gly, Gly-Gly, Ala, Ser, Gly-Phe, Gly-Leu-Phe, Gly-Phe-Phe, Gly-D-Phe-Phe, Ala-Gly-Val-Phe, Gly-Gly-Val-Phe, Gly-Phe-Tyr, Gly-β-Ala-Tyr, Gly-Leu, Gly-Phe-Leu-Gly, Gly-Phe-Gly, Gly-Gly, or any combination thereof. The oligopeptide can be linked by an amine, amide, ester, ether, thioether, acyl hydrazones, carbonate, carbamate, disulfide linkage and alike.

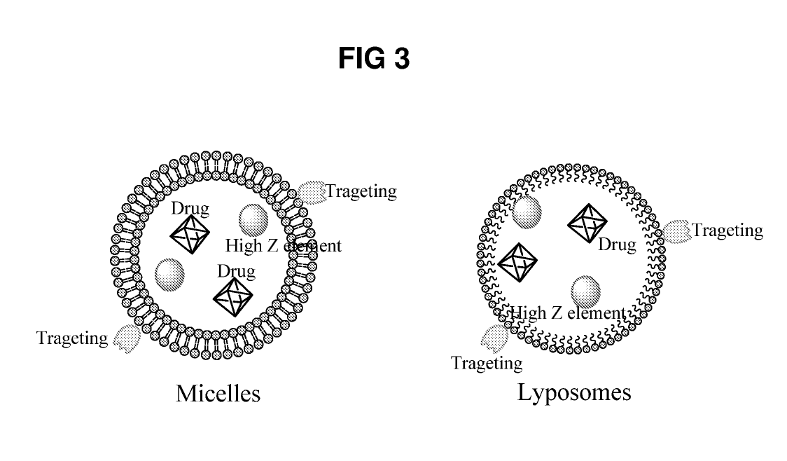

In other aspects, the macromolecule can be an amphiphile. Amphiphiles useful herein are compounds possessing hydrophilic and lipophilic groups capable of forming micelles or liposomes. The amphiphiles should be biocompatible such that they possess minimal toxicity. Amphiphiles useful herein for preparing liposomes and micelles include homopolymers, copolymers, block-copolymers produced from biocompatible and biodegradable materials. Examples of such macromolecules include, but are not limited to, poly(amino acid)s; polylactides; poly(ethyleneimine)s; poly(dimethylaminoethylmethacrylate)s, copolymers of polyethyelene glycol and hydroxyalkyl acrylates and acrylamides (e.g., N-(2-hydroxypropyl)methacrylamide), PEG-α-poly(α-amino acids), poly(L-lactic acid)-poly(ethylene glycol) block copolymers, or poly(L-histidine)-poly(ethylene glycol) block copolymers. FIG. 3 shows a polymer in the form of micelles or liposomes that can entrap drugs and high Z elements with a targeting group attached to the surface.

In one aspect, the amphiphile is a poloxamer. In one aspect, the poloxamer is a nonionic triblock copolymer composed of a central hydrophobic chain of polyoxypropylene (e.g., (poly(propylene oxide)) flanked by two hydrophilic chains of polyoxyethylene (e.g., poly(ethylene oxide)). In one aspect, poloxamer has the formula

HO(C₂H₄O)_{b}(C₃H₆O)ₐ(C₂H₄O)_{b}OH

wherein a is from 10 to 100, 20 to 80, 25 to 70, or 25 to 70, or from 50 to 70; b is from 5 to 250, 10 to 225, 20 to 200, 50 to 200, 100 to 200, or 150 to 200. In another aspect, the poloxamer has a molecular weight from 2,000 to 15,000, 3,000 to 14,000, or 4,000 to 12,000. Poloxamers useful herein are sold under the tradename Pluronic® manufactured by BASF.

In other aspects, the amphiphile can be a lipid such as phospholipids, which are useful in preparing liposomes. Examples include phosphatidylethanolamine and phosphatidylcholine. In other aspects, the amphiphile includes cholesterol, a glycolipid, a fatty acid, bile acid, or a saponin.

A variety of different high Z elements that produce Auger electrons can be used herein. In one aspect, the high Z elements group includes iodine, lutenium, hafnium, tantalum, tungsten, rhenium, osmium, iridium, platinum, gold, thallium, lead, bismuth, radon, franceium, or any combination thereof. In another aspect, the high Z element group is a platinum containing chemotherapeutic agent such as, for example, cisplatin, carboplatin, oxiplatin, nedaplatin, lipoplatin, satraplatin, ZD0473, BBR3464, SPI-77, or any combination thereof. The ability of the high Z elements to produce Auger electrons with respect to killing cancer cells and treating cancer are described in detail below.

The selection of the anti-cancer agent can vary as needed. The anti-cancer agent can be cell cycle specific compounds or non-cell cycle specific compounds. Although not always the case, the anti-cancer agent kills cells via a different mechanism than the high Z elements group (i.e., generation of Auger electrons). Examples of anti-cancer agents useful herein include, but are not limited to, abarelix, aldesleukin, alemtuzumab, alitretinoin, allopurinol, altretamine, amifostine, anakinra, anastrozole, arsenic trioxide, asparaginase, azacitidine, bevacizumab, bexarotene, bleomycin, bortezombi, busulfan, calusterone, capecitabine, carmustine, celecoxib, cetuximab, cladribine, cyclophosphamide, cytarabine, carmustine, celecoxib, cetuximab, cladribine, cyclophosphamide, cytarabine, dacarbazine, dactinomycin, actinomycin, dateparin, darbepoetin, dasatinib, daunomycin, decitabine, denileukin, diftitox, dexrazoxane, docetaxel, doxorubicin, dromostanolone, eculizumab, epirubicin, epoetin, erlotinib, estramustine, etoposide, exemestane, fentanyl, filgrastim, floxuridine, 5-FU, fulvestrant, gefitinib, gemcitabine, gem tuzumab, ozogamicin, goserelin, histrelin, hydroxyurea, ibritumomab, tiuxetan, idarubicin, ifosfamide, imatinib, irinotecan, lapatinib, lenalidomide, letrozole, leucovorin, leuprolide, levamisole, lomustine, CCNU, meclorethamine, megestrol, melphalan, L-PAM, mercaptopurine, 6-MP, mesna, methotrexate, mitomycin C, mitotane, mitoxantrone, nadrolone, nelarabine, nofetumomab, oprelvekin, pegasparagase, pegfilgrastim, peginterferon alpha-2b, pemetrexed, pentostatin, pipobrman, plicamycin, mithramycin, porfimer, procarbazine, quinacrine, rasburicase, rituximab, sargramostim, sorafenib, streptozocin, sunitinib, talc, tamoxifen, temozolomide, teniposide, VM-26, testolactone, thalidomide, thioguanine, 6-thioguanine, thiotepa, topotecan, toremifene, tositumomab, trastuzumab, tretinoin, ATRA, Uracil Mustard, valrubicin, vinorelbine, vorinostat, zoledronate, zoledronic acid, or an analog thereof. Analogs of any of the anti-cancer agents are also contemplated herein. For example, different derivatives of the agent can be used.

In certain aspects, the compounds described can have one or more targeting groups directly or indirectly bonded to the macromolecule. In the case when the targeting group is bonded to the macromolecule, any of the linkers described herein can be used. The selection of the targeting group can vary depending upon the mechanism of localization into the tumor cells. For example, “active” mechanisms may encompass receptor mediated targeting of the compounds described herein to a tumor cell. In the case of “passive” targeting, the targeting group can facilitate tumor localization by the EPR effect. Examples of targeting groups useful herein include, but are not limited to, monoclonal antibodies, peptides, somatostatin analogs, folic acid derivatives, lectins, polyanionic polysaccharides, or any combination thereof.

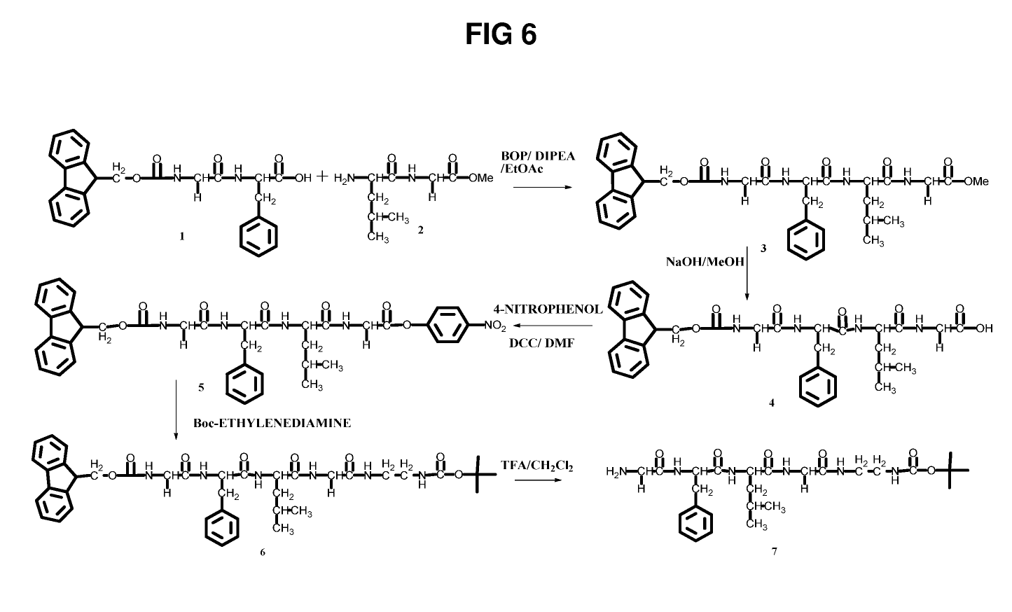

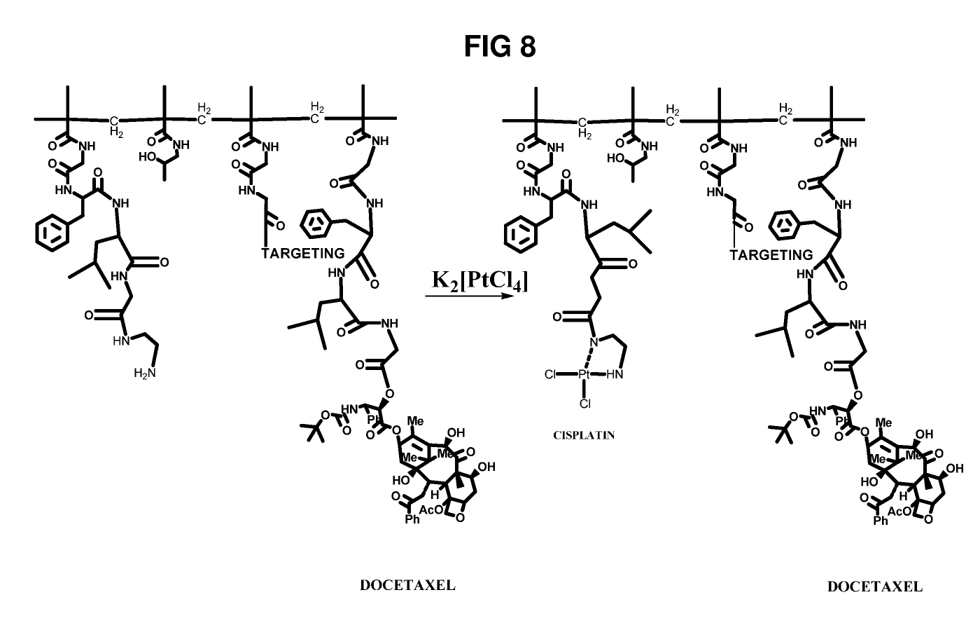

The compounds described herein can be synthesized using a variety of different synthetic approaches and techniques. Prophetic synthetic procedures are provided in the Examples below and FIGS. 6-8. In one approach, the anti-cancer agent with a linker (L-AC) and an oligopeptide (L-Oligo) ultimately used to bond the high Z elements can be synthesized independent of one another. L-AC and L-Oligo can then be sequentially covalently bonded to the macromolecule in any order. Here, the anti-cancer agent is indirectly bonded to the macromolecule. Finally, a high Z elements compound such as a salt can be reacted with the oligopeptide portion of the compound so that the high Z elements bonds (i.e., coordinates) to one or more amino acids present in the oligopeptide linker.

The compounds described herein can be formulated into a variety of pharmaceutical compositions depending upon the mode of administration. Pharmaceutical compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol.

The pharmaceutical composition can be administered in a number of ways depending on whether local or systemic treatment is desired, and on the area to be treated. Administration can be topically, including ophthalmically, vaginally, rectally, intranasally. Administration can also be intravenously or intraperitoneally. In the case of contacting cancer cells with the compounds described herein, it is possible to contact the cells in vivo or ex vivo.

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Dosing is dependent on severity and responsiveness of the condition to be treated, but will normally be one or more doses per day, with course of treatment lasting from several days to several months or until one of ordinary skill in the art determines the delivery should cease. Persons of ordinary skill can easily determine optimum dosages, dosing methodologies and repetition rates. It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

The compounds described herein can reduce or prevent tumor cell proliferation. The methods generally involve (1) contacting the tumor cells with an effective amount of a compound described herein and (2) exposing the tumor to X-ray energy at a sufficient energy to produce an Auger electron. Each step is described in detail below.

As described above, the tumor or cancer cells can be contacted with the compounds described herein in vitro, in vivo, or ex vivo. In one aspect, when the application is in vivo, the compound can be administered to a subject by techniques known in the art. For example, the compound can be administered intraveneously to the subject. Alternatively, the compound can be injected directly into the tumor. The number of times the compound is administered to the subject and the intervals of administration can vary depending upon the subject and the dosage of compound.

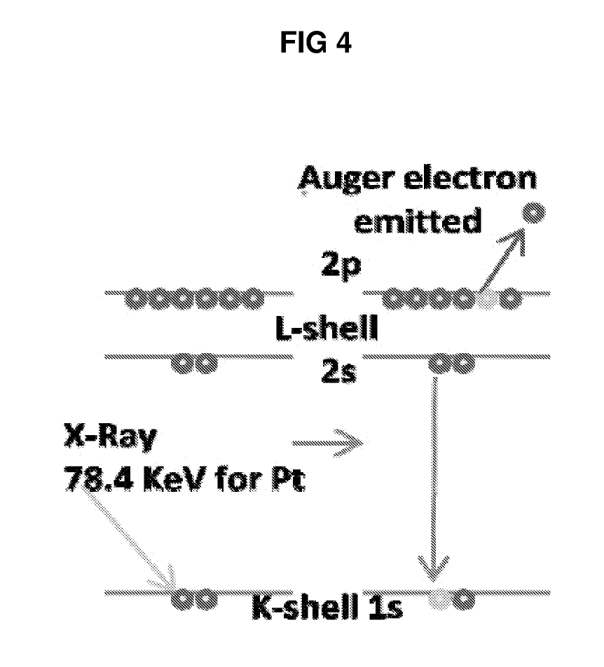

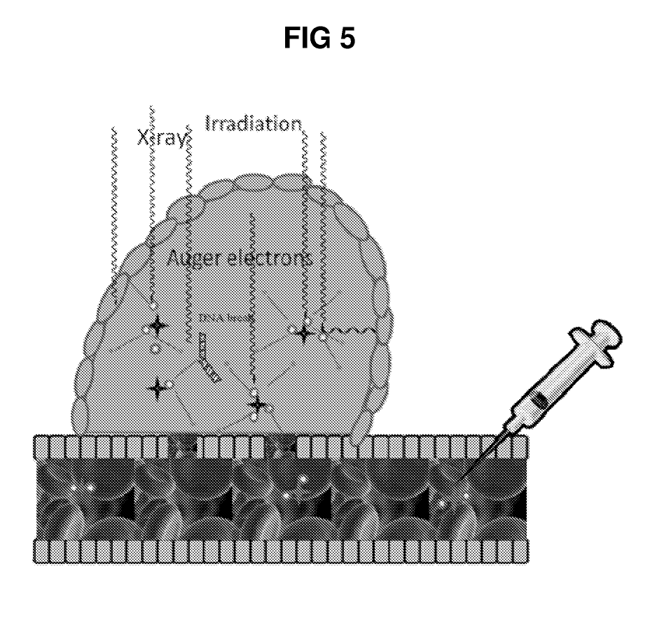

After contacting the cancer cells as described above, the tumor is exposed to X-ray energy. In certain aspects, the methods of administering X-ray energy as described herein depend on the location of the tumor. For example, it may be desirable to administer the X-ray in the entire peritoneal region (the incident beam of X-ray does not cause any significant damage as it has low energy) to patients with gynecological malignancies in the peritoneal cavity. Macromolecular delivery of the high Z elements and the anticancer agent will ensure selective accumulation of the agent in the tumor. In another aspect, the patient may have breast or testicular cancer. Upon selective accumulation of the drug and the high Z elements in these patients, localized X-ray irradiation can be administered because of low penetration depth of the tissues. In each of these aspects, upon irradiating with a specific amount of X-ray energy, the high Z elements present in the compound produces an “Auger effect.” The Auger effect is a phenomenon in which the emission of an electron from the lower orbitals of an atom causes the emission of a second electron at a higher energy orbital (see FIG. 4). For example, when an electron is removed or displaced from an inner shell of the high Z elements, an electron from a higher energy level may fall into the orbital and fill the vacancy. This results in a release of energy. This energy may be released in the form of an emitted photon or may be transferred to another electron. When the compounds described herein are present in tumor cells, the Auger effect described above also occurs. The release of energy due to the Auger effect within the tumor cell results in localized cellular damage such as single-stranded and double stranded DNA breaks, which in turn may lead to cellular death of the tumor (FIG. 5).

In conventional tumor therapies, high energy X-rays are used for radiation therapy or as a supplement to chemotherapy. While effective, high energy X-ray also damages surrounding healthy cells and tissues. However, not wishing to be bound by theory, it is contemplated that the use of low energy diagnostic X-rays in combination with the compounds described herein will minimize the damage to healthy tissues and cells during cancer treatment. This is due mainly to the fact that the production of Auger electrons requires energy levels that are substantially lower when compared to energy levels typically used in cancer radiation therapy. For example, typical radiotherapy uses therapeutic X-ray energy in the million electron volt range (MeV), where the energy required to produce Auger electrons in high Z elements is in the kilo electron volt range.

In one aspect, the cancer cells can be exposed to low energy X-ray so that the high Z elements' K shell's K-edge electrons, the L shell's L-edge electrons, or the M shell's M-edge electrons are displaced. This results in the generation of high energy in a localized space, thereby causing localized destruction within the cancer cell. For example, after the cancer cells have been contacted with a compound described herein, wherein the high Z elements is platinum, the target tumor cells can be irradiated with low energy radiation (78.4 KeV +/−2 KeV), which is the energy required to release a K shell electron. Referring to FIG. 6, exposing platinum to 78.4 KeV +/−2 KeV produces an Auger effect, wherein an electron is knocked out of the K-shell or is orbital. Subsequently an L-shell or a 2s orbital electron drops to the K-shell to occupy the displaced electron, and the difference in energy removes an outer electron (i.e., Auger electron) from the 2p orbital. The Auger electron can subsequently cause localized cellular damage within the cell as described above. Table 1 below provides the energy required to remove K shell, L shell, and M shell electrons from a number of different high Z elements useful herein. It is important to note that all of the energy levels are in kilo electron volts, which is again substantially lower than traditional radiotherapy.

TABLE 1

| | K shell | | | |
|---|---|---|---|---|
| Element | (KeV) | L_{I} (KeV) | L_{II} (KeV) | L_{III} (KeV) |
| Yb | 61.3 | 10.4 | 9.7 | 8.9 |
| Ta | 67.4 | 11.6 | 11.1 | 9.8 |
| W | 69.5 | 12.1 | 11.5 | 10.2 |
| Pt | 78.3 | 13.8 | 13.2 | 11.5 |
| Au (gold) | 80.7 | 14.3 | 13.7 | 11.9 |
| Hg | 83.1 | 14.8 | 14.2 | 12.2 |
| Tl | 85.5 | 15.3 | 14.6 | 12.6 |
| Pb | 88.0 | 15.8 | 15.2 | 13.0 |
| Bi | 90.5 | 16.3 | 15.7 | 13.4 |
| Po | 93.1 | 16.9 | 16.2 | 13.8 |
| At | 95.7 | 17.4 | 16.7 | 14.2 |

The methods described herein can be used to treat a variety of different tumors and cancers including, but not limited to, a breast tumor, a testicular tumor, an ovarian tumor, a lymphoma, leukemia, a solid tissue carcinoma, a squamous cell carcinoma, an adenocarcinoma, a sarcoma, a glioma, a blastoma, a neuroblastoma, a plasmacytoma, a histiocytoma, an adenoma, a hypoxic tumor, a myeloma, a metastatic cancer, and AIDS-related lymphoma or sarcoma, mycosis fungoides, Hodgkin's Disease, myeloid leukemia, bladder cancer, brain cancer, nervous system cancer, head and neck cancer, squamous cell carcinoma of head and neck, kidney cancer, lung cancers including small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancer, colorectal cancers, prostatic cancer, or pancreatic cancer. The choice of the high Z element, the anticancer drug and the energy would vary according to the type of cancer. The amount of energy applied to the cancer cells can vary not only with the high Z element selected but the type of cancer cell that is to be treated. For example, while Auger electrons derived from the K-shell can be used for most forms of solid tumors, the L shell energy levels can be used for cancer of the breast, testicular, head and neck and of the extremities, where the penetration depth is low. For example the choice of drugs for testicular cancer will be Docetaxel with cisplatin and the energy used will be 13.8 KeV (energy lower than that used in mammogram) while in the case of breast cancer one can choose from gemcitabin or Docetaxel and gold nano particle and X-ray energy of 14.3 KeV. In addition to killing cancer cells by the in situ generation of Auger electrons, the presence of the anti-cancer agent present in the compounds described herein provides a second mechanism for killing cancer cells. Thus, the compounds described herein provide a potent way to kill cancer cells and provide an effective treatment for cancer.

### EXAMPLES

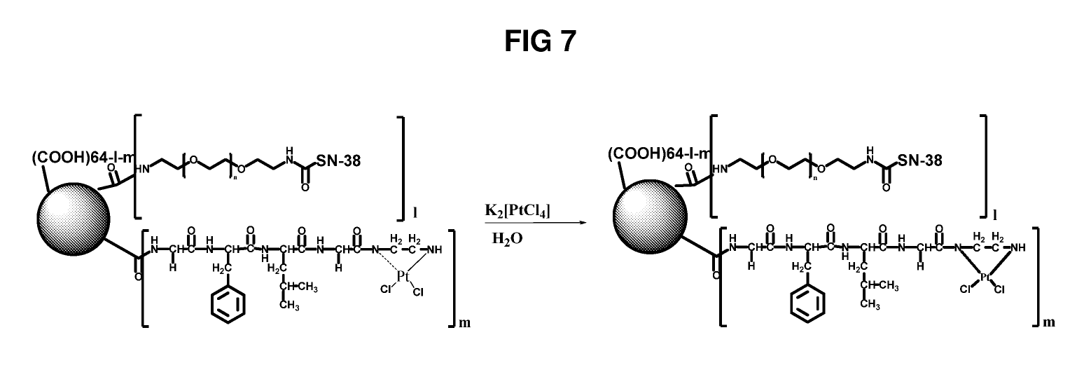

The following prophetic examples are put forth so as to provide those of ordinary skill how to make exemplary compounds described herein. FIGS. 6 and 7 are reaction schemes for the prophetic methods provided below.

Synthesis of HPMA-docetaxel-DTPA-Bi-RGDfK: The synthesis of comonomers HPMA, MA-GFLG-OH, MA-GFLG-ONp, MA-GG-ONp were carried out as described in literature (Borgman et. al. Journal of Controlled Release 132, 193-199 (2008), Pharm Res (2009)) with modification. N-(2-hydroxypropyl)methacrylamide (HPMA) (m.p. 66-68° C., MW 143.8); reactive ester comonomer, N-methacryloylglycylglycyl-p-nitrophenyl ester (MA-GG-ONp) (ε₂₇₃=9280.7 M⁻¹ cm⁻¹, m.p. 160-163° C., MW 321.7) and Bi chelating comonomer, N-methacryloylaminopropyl-2-amino-3-(isothiourea-phenyl) propyl-cyclohexane-1,2-diamine-N,N—N′,N′,N″,N″-pentaacetic acid (APMA-CHX-A″-DTPA) (ε₂₇₄=5114.6 M⁻¹ cm⁻¹, MW 736.7) were synthesized and characterized according to previously described methods. N-methacryloylglycylglycyl-RGDfK (MA-GG-RGDfK, MW 786.9) was synthesized via p-nitrophenyl ester aminolysis of MA-GG-ONp in dry DMF in the presence of pyridine for 48 h. The product was monitored by UV spectrophotometry (λ=220), and elution peaks pooled and lyophilized. The synthesis of MA-GFLG-docetaxel was carried out by reacting MA-GFLG-OH and docetaxel in the presence of DIPC and DMAP.

HPMA copolymers were synthesized via free radical precipitation copolymerization of comonomers in 100% dimethyl sulfoxide (DMSO) using N,N′-azobisisobutyronitrile (AIBN) as the initiator and 15 mol % (of total monomer feed) 3-mercaptopropionic acid (MPA) as a chain transfer agent. The feed composition of the comonomers was 10 mol % for MA-GG-RGDfK, 10 mol % for APMA-CHX-A″-DTPA, and 80 mol % for HPMA. The comonomer mixtures were sealed in an ampoule under nitrogen and stirred at 50° C. for 24 h.

The polymer conjugates were labeled by chelating Bi to CHX-A″-DTPA in the side-chains using the hydrochloride solution (0.5 mL) of Bi (or any high Z element) was buffered to approximately pH 5.0 with 100 μl of 1 M sodium acetate buffer to which 2 mg of copolymer conjugate in 100 μl of sodium acetate buffer was added, followed by incubation at 50° C. for 45 min (pH 5.0) in an evacuated sterile vial. The reaction was quenched with 50 μl of 0.05 M EDTA over 10 min at 22° C. to scavenge any free Bi. The labeled conjugates were isolated in normal saline over a Sephadex G-25 (PD-10) column.

### Synthesis of PEG-Doxorubicin-GNP

Colloidal solution of carboxyl terminated PEGylated gold nanoparticles in water was added to N,N′-dimethylformamide and 1,4-Dioxane and diisopropylethyl amine followed by addition of TSTU

### Synthesis of PAMAM-SN-38-Cisplatin

Synthesis of Fmoc-GFLG-ONp (FIG. 6). H-GF-OH will be dissolved in methanol followed by the addition of pyridine. FmocOSu will be added to the reaction mixture and stirred overnight. The reaction mixture will be concentrated under vacuum followed by trituration with diethyl ether. This will be purified by column chromatography (silica gel) using ethyl acetate (EtOAc) and methanol as eluent. The fractions containing Fmoc-GF-OH will be concentrated and used in subsequent reaction without further purification. H-LG-OH will be dissolved in methanol and cooled to 0° C. followed by the addition of SOCl₂. The reaction mixture will be refluxed followed by the removal of solvent under vacuum. The product will be redissolved in toluene and concentrated under vacuum to remove traces of methanol and SOCl₂. The solid (LGOMe) will be dissolved in EtOAc and Fmoc-GF-OH will be added to it followed by the addition of DIPEA and BOP and stirred overnight. The solvent will be removed under vacuum and washed with H₂O and extracted with EtOAc. The product will be purified by column chromatography (silica gel, eluent EtOAc: methanol; 9:1 v/v). The fraction containing Fmoc-GFLG-OMe will be concentrated under vacuum. Fmoc-GFLG-OMe will be dissolved in methanol and aqueous 1N NaOH will be added dropwise at room temperature and stirred for 6 hours. The reaction mixture will be concentrated to 50% the volume and the pH of the solution will be adjusted to pH 3. The precipitate will be extracted with EtOAc. The EtOAc layer will be concentrated to give Fmoc-GFLG-OH. The product will be identified by mass spectrometry. Fmoc-GFLG-OH will be dissolved in DMF (anhydrous) and cooled in an ice bath to −15° C., an excess of DCC solution will be added dropwise with constant stirring followed by an excess of p-nitrophenol (ONp) solution. The reaction mixture will be stirred for 3 h at −15° C., followed by overnight at 4° C. and an additional 24 h at room temperature. Trace amounts of acetic acid (glacial) will be added to the solution to react with excess DCC. The precipitated byproduct DCU will be removed and the filtrate concentrated to obtain the product (Fmoc-GFLG-ONp). Recrystallization will be done from EtOH/H₂O 1:1 (v/v). Fmoc-GFLG-ONp will be dissolved in DCM followed by the addition of bocethylenediamine and TEA. The reaction mixture will be stirred and then concentrated followed by purification by column chromatography (silica gel, eluent EtOAc). The fractions containing Fmoc-GFLG-ED-Boc was concentrated and analyzed by mass spectroscopy.

Synthesis of PEG-SN38. SN38 (AK Scientifi, Inc., Mountain View, Calif.) was conjugated as described in literature (Bioorg Med Chem 1998, 6 (5), 551-62) with slight modification. SN38 was dissolved in CH₂Cl₂/THF and treated with dit-butyl dicarbonate, and pyridine and stirred at room temperature for 6 h to selectively protect 10-hydroxy group of SN38. The product will then be treated with boc glycine followed by DMAP, and DIPC was added dropwise at 0° C. The stirring was continued for 6 hours at 0° C. and then at room temperature overnight. The reaction mixture was concentrated, treated with TFA, vortexed for 5 min. The product was neutralized with diisopropylethylamine followed by the treatment of tert-butyldimethylsilylchloride (TBDMS-Cl). The product was coupled with PEG using EDC and DMAP in DMF/DCM. The solvent was removed and purified by PD-10 column to yield PEG-SN38.
Synthesis of PAMAM dendrimer-GFLG-ethylenediamine boc-PEG-SN38 conjugates. Methanolic G3.5 dendrimer was concentrated under vacuum, reconstituted in water and the pH adjusted to 3 with dilute HCl. To this was added N-hydroxysulfosuccinimide followed by EDCI. After 2 hours GFLG-EDBoc as well as PEGylated SN38 was added and stirred overnight. The reaction mixture was lyophilized, reconstituted and dialysed to give the desired product. The product was analysed by FPLC for number and weight average molecular weight, UV for drug content and MALDI TOF for mass of the molecule.
Synthesis of dendrimer-GFLG-ethylenediamine-Pt-GFLG-SN38 conjugates (FIG. 7). Dendrimer-GFLG-ethylenediamine-boc-GFLG-SN38 was treated with TFA and vortexed for 5 mins. The product was triturated with cold acetone and the solvent removed under vacuum. Amine content was determined by ninhydrin method. Briefly 1 ml of a solution of 20 g/L ninhydrin and 3 g/L hydrintin in DMSO and sodium acetate buffer (pH 5.5, 75:25 v/v) was added to 1 mL solution of sample and incubated at 75° C. for 15 minutes and another 15 minutes at room temperature followed by addition of 3 ml ethanol (50% v/v in double distilled water). The absorbance was recorded at 570 nm and compared with the standard curve drawn against boc-ethylene diamine. The product was dissolved in DDW and K₂[PtCl₄] dissolved in DDW was added dropwise over 20 minutes. Unbound platinum was removed by centrifugation (Amincon “CENTRIPREP” filters) spinning at 2000×g for 40 minutes (minimum of 4 times). The product was lyophilized and stored at 4° C.
Synthesis of dendrimer-GFLOH-GFLG-SN38 conjugates. The mixture was dried, dissolved in methanol and filtered using membrane filter. The filtrate was dried under vacuum pump, dialyzed, and freeze dried for further characterization. All monomers was characterized by mass spectrometry and NMR while weight average molecular weight (M_{w}), number average molecular weight (Mₙ) and polydispersity (M_{w}/Mₙ) of the polymeric conjugates was estimated using size exclusion chromatography (SEC).
Synthesis of dendrimer-GFLG-OPt-GFLG-SN38 conjugates. Dendrimer-GFLGOH-PEG-SN38 was dissolved in double distilled water and molar equivalent of NaHCO₃ was added and stirred for 30 minutes to give dendrimer-GFLG-ONa-PEG-SN38. Pt(NH₃)₂Cl₂ was dissolved in DDW and added dropwise over 20 minutes to the solution of dendrimer-GFLG-ONa-PEG-SN38 with stirring at room temperature. Unbound platinum was removed by centrifugation (Amincon “CENTRIPREP” filters) spinning at 2000 g for 40 minutes (minimum of 4 times) and product lyophilized.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein were apparent from consideration of the specification and practice of the compounds, compositions and methods disclosed herein. It is intended that the specification and examples be considered as exemplary.

1. A compound comprising a macromolecule comprising (1) at least one anti-cancer agent directly or indirectly bonded to the macromolecule and (2) at least one high Z element directly or indirectly bonded to the macromolecule that is capable of producing Auger electrons upon exposure to X-ray energy.

2. The compound of claim 1, wherein the compound comprises the formula I 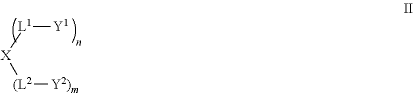
wherein
X is a macromolecule;
L¹ and L² are a linker;
Y¹ comprises a high Z element group bonded to the linker L¹;
Y² comprises an anti-cancer agent covalently bonded to the linker L²;
and m and n are, independently, an integer from 1 to 10.

3. The compound of claim 1, wherein the macromolecule comprises dextran, dextrin, hyaluronic acid, chitosan, polylactic/glycolic acid (PLGA), poly lactic acid (PLA), polyglutamic acid (PGA), polymaleic acid, polyaspartamides, polyethylene glycol (PEG N-(2-hydroxypropyl)methacrylamide (HPMA), polyvinylpyrrolidone, polyethyleneimine, polyamidoamine (linear), poly(amido amine) (PAMAM), diaminobutane (DAB), diaminoethane (DAE), and a dendrimer comprising polyamidoamine, polypropyleneimine, polyether, polylysine, or any combination thereof.

4. The compound of claim 1, wherein the macromolecule comprises a macromolecule derived from the polymerization of one or more monomers comprising N-(2-methylpropyl)methacrylamide, N-(2-methylethyl)methacrylamide, N-isopropyl methacrylamide, N,N-dimethacrylamide, N-vinylpyrrolidone, vinyl acetate, 2-methacryloxyethyl glycoside, acrylic acid, methacrylic acid, vinylphosphonic acid, styrenesulfonic acid, malic acid, 2-methacryloxyethyltrimethylammonium chloride, 2-methacrylamidopropyltrimethylammonium chloride, methacryloylcholine methyl sulfate, 2-methacryloxyethyltrimethylammonium bromide, 2-venyl-1-methylpyridinium bromide, 4-vinyl-1-methylpyridinium bromide, ethyleneimine, (N-acetyl)ethyleneimine, (N-hydroxyethyl)ethyleneimines, or allylamine.

5. The compound of claim 3, wherein the dendrimers comprises a range of 1 to 10 generations or half generations.

6. The compound of claim 1, wherein the macromolecule has a molecular weight of 10 kD to 200 kD.

7. The compound of claim 1, wherein the high Z element group comprises iodine, lutenium, hafnium, tantalum, tungsten, rhenium, osmium, iridium, gold, thallium, lead, bismuth, radon, franceium, platinum or any combination thereof.

8. The compound of claim 1, wherein the high Z element group comprises platinum containing chemotherapeutic agent.

9. The compound of claim 7, wherein the platinum containing chemotherapeutic agent comprises cisplatin, carboplatin, oxiplatin, nedaplatin, lipoplatin, satraplatin, ZD0473, BBR3464, SPI-77, or any combination thereof.

10. The compound of claim 1, wherein the anti-cancer agent comprises a cell cycle specific compound.

11. The compound of claim 1, wherein at least one anti-cancer agent comprises abarelix, aldesleukin, alemtuzumab, alitretinoin, allopurinol, altretamine, amifostine, anakinra, anastrozole, arsenic trioxide, asparaginase, azacitidine, bevacizumab, bexarotene, bleomycin, bortezombi, busulfan, calusterone, capecitabine, carmustine, celecoxib, cetuximab, cladribine, cyclophosphamide, cytarabine, carmustine, celecoxib, cetuximab, cladribine, cyclophosphamide, cytarabine, dacarbazine, dactinomycin, actinomycin, dateparin, darbepoetin, dasatinib, daunomycin, decitabine, denileukin, diftitox, dexrazoxane, docetaxel, doxorubicin, dromostanolone, eculizumab, epirubicin, epoetin, erlotinib, estramustine, etoposide, exemestane, fentanyl, filgrastim, floxuridine, 5-FU, fulvestrant, gefitinib, gemcitabine, gemtuzumab, ozogamicin, goserelin, histrelin, hydroxyurea, ibritumomab, tiuxetan, idarubicin, ifosfamide, imatinib, irinotecan, lapatinib, lenalidomide, letrozole, leucovorin, leuprolide, levamisole, lomustine, CCNU, meclorethamine, megestrol, melphalan, L-PAM, mercaptopurine, 6-MP, mesna, methotrexate, mitomycin C, mitotane, mitoxantrone, nadrolone, nelarabine, nofetumomab, oprelvekin, pegasparagase, pegfilgrastim, peginterferon alpha-2b, pemetrexed, pentostatin, pipobrman, plicamycin, mithramycin, porfimer, procarbazine, quinacrine, rasburicase, rituximab, sargramostim, sorafenib, streptozocin, sunitinib, talc, tamoxifen, temozolomide, teniposide, VM-26, testolactone, thalidomide, thioguanine, 6-thioguanine, thiotepa, topotecan, toremifene, tositumomab, trastuzumab, tretinoin, ATRA, Uracil Mustard, valrubicin, vinorelbine, vorinostat, zoledronate, zoledronic acid, or an analog thereof.

12. The compound of claim 2, wherein L¹ and L² comprises, independently, an oligopeptide sequence, an amino acid or amino acid sequence, polyethylene glycol, (PEG-diacrylate (PEGDA), PEG-dimethacrylate (PEGDM), PEG-diacrylamide (PEGDAA), or PEG-dimethacrylamide (PEGDMA).

13. The compound of claim 12, wherein the amino acid or amino acid sequence comprises Gly-Ileu-Phe, Gly-Val-Phe, Gly-Gly-Phe, Gly-Gly-Phe-Phe, Gly-Ileu-Tyr, Gly-Gly, Gly-Phe, Gly-Leu-Phe, Gly-Phe-Phe, Gly-D-Phe-Phe, Ala-Gly-Val-Phe, Gly-Gly-Val-Phe, Gly-Phe-Tyr, Gly-B-Ala-Tyr, Gly-Leu, Gly-Phe-Leu-Gly, Gly-Phe-Gly, Gly-Gly, Phe, Gly, Ala, Ser, or any combination thereof.

14. The compound of claim 12, wherein the polyethylene glycol has a molecular weight from 62 D to 20 kD.

15. The compound of claim 1, wherein the compound further comprises a targeting group directly or indirectly bonded to the macromolecule.

16. The compound of claim 1, wherein the targeting group is covalently bonded to the linker.

17. The compound of claim 15, wherein the targeting group comprises a monoclonal antibody, a peptide, a somatostatin analog, a folic acid derivative, a lectin, a polyanionic polysaccharide, or any combination thereof.

18. The compound in claim 15, wherein the targeting group is indirectly bonded to the macromolecule by a linker.

19. The compound of claim 2, wherein X comprises a poly (amido amine) dendrimer, L¹ comprises one or more amino acids, and L² comprises polyethylene glycol.

20. The compound of claim 19, wherein Y¹ comprises a cisplatin group.

21. The compound of claim 20, wherein Y² comprises a camptothecin or an analog thereof.

22. A method of reducing or preventing tumor cell proliferation comprising (1) contacting the tumor cells with an effective amount of a compound of claim 1 and (2) exposing the cells to X-ray energy at a sufficient level to produce an Auger electron.

23. The method of claim 22, wherein the X-ray energy is sufficient to remove a K-shell electron from the high Z element.

24. The method of claim 22, wherein the X-ray energy is sufficient to remove a L-shell electron from the high Z element.

25. The method of claim 22, wherein the tumor comprises a breast tumor, a testicular tumor, an ovarian tumor, a lymphoma, leukemia, a solid tissue carcinoma, a squamous cell carcinoma, an adenocarcinoma, a sarcoma, a glioma, a blastoma, a neuroblastoma, a plasmacytoma, a histiocytoma, an adenoma, a hypoxic tumor, a myeloma, a metastatic cancer, and AIDS-related lymphoma or sarcoma, mycosis fungoides, Hodgkin's Disease, myeloid leukemia, bladder cancer, brain cancer, nervous system cancer, head and neck cancer, squamous cell carcinoma of head and neck, kidney cancer, lung cancers including small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancer, colorectal cancers, prostatic cancer, or pancreatic cancer.
